(19) United States
(12) Patent Application Publication
(10) Pub. No.: US 2023/0223125 A1
(43) Pub. Date: Jul. 13, 2023

(54) ARTIFICIAL INTELLIGENCE-BASED SYSTEMS AND METHODS FOR DOSING OF PHARMACOLOGIC AGENTS

(71) Applicant: University of Louisville Research Foundation, Inc., Louisville, KY (US)

(72) Inventors: Adam E. Gaweda, Jeffersonville, IN (US); Michael E. Brier, Rossville, TN (US); Eleanor D. Lederer, Louisville, KY (US)

(73) Assignees: University of Louisville Research Foundation, Inc., Louisville, KY (US); United States Department of Veterans Affairs, Washington, DC (US)

(21) Appl. No.: 17/997,667

(22) PCT Filed: May 4, 2021
(86) PCT No.: PCT/US2021/030627

§ 371 Date: Nov. 1, 2022

Related U.S. Application Data
(60) Provisional application No. 63/019,973, filed on May 4, 2020.

(51) Int. Cl. G16H 20/10; G06N 3/08

(52) U.S. Cl. CPC G16H 20/10; G06N 3/08

(57) ABSTRACT

The present invention relates to systems and methods for personalized dosing of pharmacologic agents. In particular, the presently-disclosed subject matter relates to a computer-based system and method for personalized dosing of one or more pharmacologic agents to optimize one or more therapeutic responses. In some embodiments, the computer-based system and method provides for a computer-based model of a complex biological system useful for training machine learning agents for optimizing personalized dosing of pharmacological agents.

### CROSS-REFERENCE TO RELATED APPLICATION

This application claims the benefit of priority to U.S. provisional patent application Ser. No. 63/019,973, filed May 4, 2020, for SYSTEMS AND METHODS FOR PERSONALIZED ADMINISTRATION OF PHARMACOLOGIC AGENTS, incorporated herein by reference.

### GOVERNMENT LICENSE RIGHTS

This application was made with government support under Grant 1CX001614 awarded by the Department of Veterans Affairs, Veterans Health Administration, Office of Research and Development. The government has certain rights in the invention.

### FIELD OF THE INVENTION

The present invention relates to systems and methods for personalized dosing of pharmacologic agents. In particular, the presently-disclosed subject matter relates to a computer-based system and method for personalized dosing of one or more pharmacologic agents to optimize one or more therapeutic responses. In some embodiments, the computer-based system and method provides for a computer-based model of a complex biological system useful for training machine learning agents for optimizing personalized dosing of pharmacological agents.

### BACKGROUND OF THE INVENTION

Numerous diseases and disorders are effectively treated and/or managed by administering various pharmacologic agents to the patients that are suffering from those diseases and disorders. In this regard, and to provide standardized care for these subjects, a number of dosing regimens and guidelines have been developed for each of the various pharmacologic agents. However, these dosing regimens and guidelines have often overlooked not only the complexity of the diseases and disorders being treated by the pharmacologic agents, but the dosing regimens and guidelines have also often overlooked the variability in the responses each particular patient may have to a particular pharmacologic agent, which may lead to an adverse outcome.

For example, chronic kidney disease mineral bone disorder (CKD-MBD) is a common complication of kidney diseases, starting early in the course of disease and resulting in devastating clinical consequences ranging from bone fragility to accelerated atherosclerosis and early cardiovascular death. CKD-MBD involves phosphate accumulation leading to the development of secondary hyper-parathyroidism, elevated fibroblast growth factor 23 (FGF23) levels, decreased calcitriol (CTL) levels, disordered bone metabolism, and vascular calcification. Observational data in patients with CKD show a doubling in mortality as phosphate concentrations increase from below 5 mg/dL to above 9 mg/dL. An additional consequence of CKD-MBD is the increased risk of fracture and an increased mortality due to that fracture. Patients with a glomerular filtration rate (GFR)<60 mL/min were at a two-fold risk of developing hip fractures.

In 2003, the U.S. National Kidney Foundation published The Kidney Disease Outcomes Quality Initiative (K/DOQI) guidelines that established target levels for calcium (Ca), phosphorous (P), and parathyroid hormone (PTH) attempting to lessen the impact of CKD-MBD on mortality. Attainment of these goals has been shown to lessen the impact of CKD-MBD on mortality. However, incomplete understanding of CKD-MBD, individual variability in the manifestations of CKD-MBD, and the need for multiple pharmacologic agents to treat this disease have made it difficult to maintain these targets for Ca, P, and PTH.

Accordingly, a system and method for dosing pharmacologic agents that allows more precise control over the dosing of the pharmacological agents, while also taking into account the variability among subjects and their responses, would be both highly desirable and beneficial.

### SUMMARY

The instant subject matter relates to systems and methods for personalized dosing of pharmacological agents. This group has previously demonstrated that computational methods based on combination of mathematical modeling, feedback control, and machine learning can be successfully employed in treatment of anemia arising from CKD, as described in U.S. Pat. Nos. 9,852,267 and 10,803,142, both of which are incorporated herein by reference. The instant subject matter expands the application of machine learning in kidney disease to the management of CKD-MBD through the creation of a mathematical model describing the CKD-MBD progression effect on clinical markers of mineral metabolism and their response to treatment. One application of the proposed model is for development of personalized pharmacologic and non-pharmacologic therapeutic regimens of CKD-MBD. Furthermore, the model is easily adaptable, such that new therapies, diagnostic tests, and different dialysis modalities can be easily incorporated.

In some embodiments, the present invention is a system for personalized dosing of a pharmacologic agent comprising a data storage device; a drug dosing agent stored on the data storage device, the drug dosing agent for determining a dose set for one or more pharmacologic agents; a computational model of a biological system stored on the data storage device; a reinforcement learning algorithm stored on the data storage device; and a processing device in communication with the data storage device, the processing device configured to: execute the drug dosing agent to determine the dose set for one or more pharmacologic agents; execute the computational model to simulate the effects of the dose set, the computational model generating an output physiological state; and execute the drug dosing agent to adjust the dose set for the one or more pharmacologic agents based at least in part on the output physiological state. In further embodiments, the computational model is one of a quantitative systems pharmacology model and a systems biology model. In certain embodiments, the computational model is a model of chronic kidney disease. In some embodiments, the computational model is a model of chronic kidney disease mineral bone disorder. In further embodiments, wherein the drug dosing agent is a deep neural network. In certain embodiments, the drug dosing agent is trained using reinforcement learning techniques rewarding the output physiological state achieving one or more target ranges. In some embodiments, the drug dosing agent adjusts the dose set for the one or more pharmacologic agents based in part on the output physiological state and based in part on physiological data from a subject. In further embodiments, the computational model represents the biological system as a plurality of compartments, each compartment representing a tissue or organ, including a soft tissue compartment. In certain embodiments, the computational model includes a smooth muscle cell compartment.

In some embodiments, the present invention is a method for providing personalized dosing of a pharmacologic agent to a patient, comprising: obtaining a target range for an output physiological state; determining, using a computer-implemented drug dosing agent, a dose set for a pharmacologic agent; simulating, using a computational model of a biological system, effects of administering the dose set; generating, using the computational model, the output physiological state based at least in part on the effect of the dose set; repeating the determining, simulating, and generating steps until the output physiological state is within the target range, wherein the determining is based at least in part on the output physiological state; and determining, using the computer-implemented drug dosing agent, a patient dose set for the pharmacologic agent. In further embodiments, the computational model is one of a quantitative systems pharmacology model and a systems biology model. In certain embodiments, the computational model is a model of chronic kidney disease.

In some embodiments, the computational model is a model of chronic kidney disease mineral bone disorder. In further embodiments, the drug dosing agent is a deep neural network. In certain embodiments, the output physiological state is a plurality of output physiological states, and wherein the target range is a plurality target ranges, each of the output physiological states having one target range. In some embodiments, the pharmacologic agent is one of a phosphate binder, a calcimimetic, and vitamin D and analogs and metabolic precursors thereof. In further embodiments, the output physiological state is calcium concentration and wherein the target range is 8.4 mg/dL to 10.2 mg/dL. In certain embodiments, the output physiological state is phosphorous concentration and wherein the target range is 3.5 mg/dL to 5.5 mg/dL. In some embodiments, the output physiological state is parathyroid hormone concentration and wherein the target range is 130 pg/mL to 600 pg/m L. In further embodiments, the pharmacologic agent modifies the output physiological state.

In some embodiments, the present invention is a data storage device having computer program instructions stored thereon that, when executed by a processor, cause the processor to perform the following instructions: obtaining a target range for an output physiological state; determining, using a computer-implemented drug dosing agent, a dose set for a pharmacologic agent; simulating, using a computational model of a biological system, the effect of the dose set; generating, using the computational model, the output physiological state based at least in part on the effect of the dose set; repeating the determining, simulating, and generating steps until the output physiological state is within the target range, wherein the determining is based at least in part on the output physiological state; and determining, using the computer-implemented drug dosing agent, a patient dose set for the pharmacologic agent.

In some embodiments, the present invention is a data storage device having computer program instructions stored thereon that, when executed by a processor, cause the processor to perform the following instructions: simulate progression of chronic kidney disease metabolic bone disorder in a patient, the patient being represented by a plurality of compartments, each compartment representing a tissue or organ, wherein progression of chronic kidney disease metabolic disorder is simulated changes in concentrations of compounds in each compartment. In further embodiments, the compounds include at least one of fibroblast growth factor 23, calcium, phosphorous, and parathyroid hormone. In certain embodiments, the plurality of compartments include a compartment representing smooth muscle cells. In some embodiments, the plurality of compartments include a compartment representing soft tissue.

It will be appreciated that the various systems and methods described in this summary section, as well as elsewhere in this application, can be expressed as a large number of different combinations and subcombinations. All such useful, novel, and inventive combinations and subcombinations are contemplated herein, it being recognized that the explicit expression of each of these combinations is unnecessary.

### BRIEF DESCRIPTION OF THE DRAWINGS

A better understanding of the present invention will be had upon reference to the following description in conjunction with the accompanying drawings.

FIG. 1 depicts a schematic diagram of the Quantitative Systems Pharmacology model of Chronic Kidney Disease — Metabolic Bone Disorder.

FIG. 2A is a graph depicting performance of the disclosed system in predicting the concentrations of P over a range of renal function (model) compared to the model published by Peterson and Riggs (base model) and actual patient data (Lima).

FIG. 2B is a graph depicting performance of the disclosed system in predicting the concentrations of Ca over a range of renal function (model) compared to the model published by Peterson and Riggs (base model) and actual patient data

(Lima).

FIG. 2C is a graph depicting performance of the disclosed system in predicting the concentrations of PTH over a range of renal function (model) compared to the model published by Peterson and Riggs (base model) and actual patient data (Lima).

FIG. 2D is a graph depicting performance of the disclosed system in predicting the concentrations of CTL over a range of renal function (model) compared to the model published by Peterson and Riggs (base model) and actual patient data (Lima).

FIG. 2E is a graph depicting performance of the disclosed system in predicting the concentrations of intact FGF23 over a range of renal function (model) compared to actual patient data (Lima).

FIG. 3A is a graph depicting performance of the disclosed system in predicting the concentrations of P over a range of renal function (model) compared to the model published by Peterson and Riggs (base model) and actual patient data (Pires).

FIG. 3B is a graph depicting performance of the disclosed system in predicting the concentrations of Ca over a range of renal function (model) compared to the model published by Peterson and Riggs (base model) and actual patient data (Pires).

FIG. 3C is a graph depicting performance of the disclosed system in predicting the concentrations of PTH over a range of renal function (model) compared to the model published by Peterson and Riggs (base model) and actual patient data (Pires).

FIG. 3D is a graph depicting performance of the disclosed system in predicting the concentrations of CTL over a range of renal function (model) compared to the model published by Peterson and Riggs (base model) and actual patient data (Pires).

FIG. 4 is a graph depicting steady-state Ca flux from serum to soft tissue (circles) and from bone to serum (squares) as predicted by the QSP model.

FIG. 5A is a graph depicting serum Ca concentration over time as predicted by the QSP model following the initiation of dialysis, and treatment with phosphate binders, vitamin D, and a calcimimetic.

FIG. 5B is a graph depicting serum P concentration over time as predicted by the QSP model following the initiation of dialysis, and treatment with phosphate binders, vitamin D, and a calcimimetic.

FIG. 5C is a graph depicting PTH concentration over time as predicted by the QSP model following the initiation of dialysis, and treatment with phosphate binders, vitamin D, and a calcimimetic.

FIG. 6 is a graph depicting Ca flux over time as predicted by the QSP model following the initiation of dialysis, and treatment with phosphate binders, vitamin D, and a calcimimetic.

FIG. 7 is a graph comparing the second embodiment model (solid line) and base model (dashed line) predictions of serum calcium concentration over a range of GFRs as compared to subject data (points).

FIG. 8 is a graph comparing the second embodiment model (solid line) and base model (dashed line) predictions of serum phosphorous concentration over a range of GFRs as compared to subject data (points).

FIG. 9 is a graph comparing the second embodiment model (solid line) and base model (dashed line) predictions of serum calcitriol concentration over a range of GFRs as compared to subject data (points).

FIG. 10 is a graph comparing the second embodiment model (solid line) and base model (dashed line) predictions of serum PTH concentration over a range of

GFRs as compared to subject data (points).

FIG. 11 is a graph depicting the second embodiment model (solid line) prediction of FGF23 concentration over a range of GFRs as compared to subject data (points).

FIG. 12A depicts a block diagram of reinforcement learning framework for off-line training of drug dosing agent.

FIG. 12B depicts a block diagram of drug dosing agent deployed with patient on-line.

FIG. 13A is a graph depicting measured patient serum calcium concentrations upon implementation of a drug dosing agent.

FIG. 13B is a graph depicting measured patient serum phosphorous concentrations upon implementation of a drug dosing agent.

FIG. 13C is a graph depicting measured patient serum PTH concentrations upon implementation of a drug dosing agent.

FIG. 13D is a graph depicting measured patient serum PTH concentrations upon implementation of a drug dosing agent for a subset of patients.

### DETAILED DESCRIPTION OF THE PREFERRED EMBODIMENTS

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the following terms are believed to be well understood by one of ordinary skill in the art, definitions are set forth to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, and in some embodiments±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The term “processing device” is used herein to describe one or more microprocessors, microcontrollers, central processing units, Digital Signal Processors (DSPs), Field-Programmable Gate Arrays (FPGAs), Application-Specific Integrated Circuits (ASICs), or the like for executing instructions stored on a data storage device.

The term “data storage device” is understood to mean physical devices (computer readable media) used to store programs (sequences of instructions) or data (e.g. program state information) on a non-transient basis for use in a computer or other digital electronic device, including primary memory used for the information in physical systems which are fast (i.e. RAM), and secondary memory, which are physical devices for program and data storage which are slow to access but offer higher memory capacity. Traditional secondary memory includes tape, magnetic disks and optical discs (CD-ROM and DVD-ROM). The term “memory” is often (but not always) associated with addressable semiconductor memory, i.e. integrated circuits consisting of silicon-based transistors, used for example as primary memory but also other purposes in computers and other digital electronic devices. Semiconductor memory includes both volatile and non-volatile memory. Examples of non-volatile memory include flash memory (sometimes used as secondary, sometimes primary computer memory) and ROM/PROM/EPROM/EEPROM memory. Examples of volatile memory include dynamic RAM memory, DRAM, and static RAM memory, SRAM.

The term “pharmacologic agent” is used herein to refer to any agent capable of effecting a measurable change in a physiological characteristic of a subject. Exemplary pharmacological agents discussed herein include, but are not limited to: phosphate binders, vitamin D and analogs thereof, calcimimetics, and chemicals that are metabolized to one of the foregoing. In some embodiments, vitamin D analogs include calcitriol, doxercalciferol, paricalcitol, calcipotriene, and ergocalciferol. In some embodiments, the term “pharmacologic agent” is used interchangeably with the term “drug.”

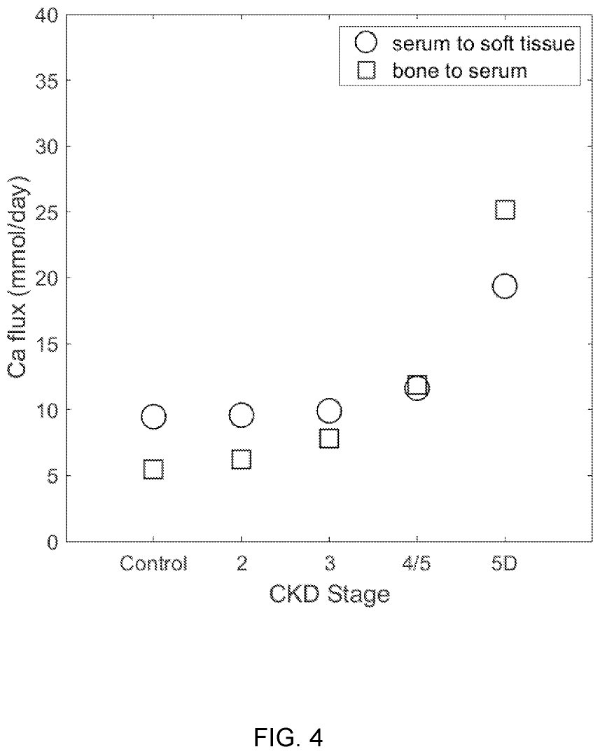

The presently-disclosed subject matter relates to a system and method for personalized dosing of pharmacological agents and, in particular, a computer-based system and method for the personalized dosing of one or more pharmacologic agents that can be used to optimize one or more therapeutic responses in a subject. In some embodiments of the presently-disclosed subject matter, the underlying methodology of a computer-based system for personalized dosing of one or more pharmacologic agents is based on the principles of quantitative systems pharmacology (QSP). In QSP, mathematical models are used to characterize biological systems, disease progression, pharmacokinetics and pharmacodynamics. Such QSP models can be used to generate new hypotheses in silico and simulate treatment strategies for new drug / indication approval. The computer-based system and method disclosed herein may, in some embodiments, be embodied in a system as shown in FIG. 4 and accompanying text of U.S. Pat. No. 9,852,267. In such embodiments, the system includes a data storage device, dosing regimen program modules (i.e., an agent) stored on the data storage device, and as described herein, a novel computational model of a biological system. The system further includes a processing device that uses the model and agent to determine a plurality of dosing regimens that map physiological or modeled responses to doses of pharmacological agent. In some embodiments, the system further includes a point-of-care device as described in U.S. Pat. No. 9,852,267.

The systems model proposed here is based on the open-source framework proposed by Peterson and Riggs, referred to herein as the “base model,” and described in Peterson M C, Riggs M M. A physiologically based mathematical model of integrated calcium homeostasis and bone remodeling. Bone 46: 49-63, 2010, incorporated herein by reference. The base model is composed of 28 differential equations describing calcium and phosphorus homeostasis and was developed for non-CKD patients. Although the model does account for the effect of progressive kidney failure, a number of important modifications were required to better match the physiology of CKD-MBD.

Parathyroid Gland Compartment

A significant manifestation of CKD-MBD is secondary hyperparathyroidism accompanied by enlargement of the parathyroid gland. To effectively model this change parathyroid cell hypertrophy, hyperplasia, and downregulation of vitamin D and calcium sensing receptors to varying degrees must be accounted for. The increase in parathyroid gland size coupled with the downregulation of negative regulatory modifiers perpetuates and enhances the clinical consequences of hyperparathyroidism in CKD. To account for the effect of P on parathyroid gland size, the equation representing parathyroid gland capacity was modified as follows:

d/dtPTG=k₁₁*H_{6.11}⁻*H_{5.11}−k₁₁*PTG

where PTG is the parathyroid gland capacity, the rate constant (k₁₁) and the Hill function H⁻_{6.11} are the same as in the base model.

H_{5.11} is a Hill function representing the effect of increase in P on the hyperplasia of the parathyroid gland:

$${H}_{S , 11} = \frac{{α}_{S , 11} \times {P}^{{γ}_{S , 11}}}{{P}^{{γ}_{S , 11}} + {δ}_{S , 11}^{{γ}_{S , 11}}}$$

The mass-balance equation describing PTH secretion was modified to account for the calcimimetic effect:

$$d / dt PTH = {H}_{4 , 10 - 7}^{-} \times ( 1 - \frac{{D}_{CM}}{{D}_{CM} + {EC}_{SOCM}} ) \times PTG - {k}_{70} \times PTH$$

where PTH is the parathyroid hormone concentration, D_{CM} is the serum concentration of the calcimimetic, and EC_{50CM} was set to 6.2 ng/m L.

FGF23

FGF23 was added to the base model to better model phosphate metabolism. FGF23 has emerged as a very important factor in physiologic mineral metabolism and in the development of multiple complications of CKD. It acts to decrease serum phosphate levels by promoting phosphaturia and decrease CTL production through the downregulation of alpha-1-hydroxylase.

Hyperphosphatemia has been found to be a major contributor to elevated FGF23 levels and this relationship is used to predict FGF23 levels in this model 29A relationship between FGF23 levels and serum phosphate concentration was determined by extracting data from pre-existing literature. The FGF23 compartment was incorporated into the base model as compartment 29 described by the following mass-balance dynamics

d/dₜFGF23=H_{5.29}⁺−0.8×FGF23

where

$${H}_{5 , 29}^{+} = {ρ}_{5 , 29} + ( {α}_{5 , 29} - {ρ}_{5 , 29} ) \times \frac{{P}^{γ 5 , 29}}{{P}^{γ 5 , 29} + {δ}_{5 , 29} γ 5 , 29}$$

The initial value of FGF23 was set at 30 pg/ml, representative of the mean value reported in healthy individuals.

Calcitriol Secretion

With the addition of FGF23 to the model, the representation of secretion of calcitriol required modification as well. The mass-balance equation describing production of 1-α-Hydroxylase (1αOH) was modified to account for downregulation by rising levels of FGF23. As above, data was extracted from literature in order to determine the relationship between FGF23 levels and CTL:

d/dₜ1αOH=k_{9.5}×H_{7.9}×H_{5.9}⁻×H_{29.9}⁻k_{9D}×1αOH+D_{CTL}

where D_{CTL} is the exogenous CTL concentration and H⁻_{29.9} is a Hill function representing the effect of FGF23 on 1αOH:

$${H}_{29 , 9}^{-} = {α}_{29 , 9} - ( {α}_{29 , 9} - {ρ}_{29 , 9} ) * \frac{FGF {23}^{{γ}_{29 , 9}}}{FGF {23}^{{γ}_{29 , 9}} + {δ}_{29 , 9}^{{γ}_{29 , 9}}}$$

Renal Phosphate Absorption

To account for the effect of FGF23 on renal phosphorous reabsorption, the urinary phosphate flux equation was modified as follows:

v_{S-u}=0.88×GFR×P−0.88×GFR×[φ_{Su}−FGF23/(FGF23+β_{Su})]

Intestinal Phosphate Absorption

Intestinal phosphate absorption was modified from the base model to simulate the administration of oral phosphate binders. The bioavailability of intestinal phosphorous (Pᵢₙₜ) is modified by CTL as well as the dose of the phosphate binder:

$$d / dt {P}_{int} = {H}_{6 , 3}^{+} * {P}_{oral} - {k}_{3 - 5} * {P}_{int} - {k}_{bnd} * {D}_{bnd} \\ {H}_{6 , 3}^{+} = 0.6 + \frac{0.3 * {CTL}^{2}}{{CTL}^{2} + {83.25}^{2}} \\ d / dt {P}_{int} = {H}_{6 , 3}^{+} \times {P}_{oral} - {k}_{3 - 5} \times {P}_{int} - {k}_{bnd} \times {D}_{bnd} \\ {H}_{6 , 3}^{+} = 0.6 + \frac{0.3 \times {CTL}^{2}}{{CTL}^{2} + {δ}_{6 , 3}^{2}}$$

where Pₒᵣₐₗ is the daily oral phosphorus intake and D_{bnd} is the daily dose of the phosphate binder and k_{bnd} is the binding capacity of the phosphate binder. The Hill function H⁺_{6.3} represents the effect of CTL on intestinal phosphate absorption, and the parameter δ_{6.3} was estimated from data presented in the literature.

Smooth Muscle Cell Compartment

The base model demonstrated a systematic deviation between observed and predicted calcium blood concentrations. In the present invention, the mismatch was rectified through the incorporation of a novel smooth muscle cell compartment (Caₛₘ, compartment 30) Calcium deposition in smooth muscle cell (Caₛₘ, compartment 30) is stimulated by the serum P and Ca concentration and described by the following equation:

d/dₜCaₛₘ=α_{5.30}×max[0,(P−δ_{5.30})^{0.55}]×Ca

The phosphorous concentration in smooth muscle cells (Pₛₘ, compartment 31) is assumed to be proportional to the smooth muscle cell Ca concentration and multiplied by the stoichiometric factor 0.464:

d/dtPₛₘ=0.464*d/dtCaₛₘ

Ca—P mass balance equations (compartments 4 and 5 in the base model) were modified as follows:

d/dtCa=v₁₂₋₄−v₄₋₁₂−v₄₋ᵤ+v₁₋−v₄₋₃₀

d/dtP=v₅₋₁₄−v₁₄₋₅−v₅₋ᵤ+v₃₋₅−v₅₋₈+v₈₋₅−v₅₋₃₁−v_{5-HD}

where v₄₋₃₀ is Ca flux between serum and soft tissue, V₅₋₃₁ is P flux between serum and soft tissue, and v_{5-HD} is P removal through dialysis.

Model Fitting

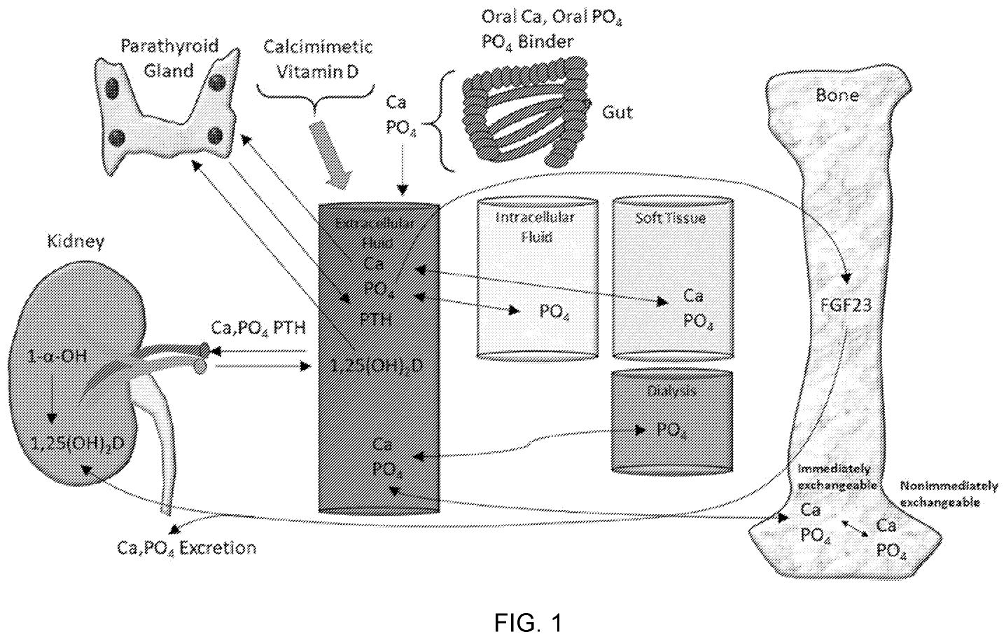

A schematic block diagram of the model is shown in FIG. 1 where arrows represent individual functions containing parameters to be estimated. In a first embodiment, parameters for the new and modified model compartments described above were fine-tuned using recently published data describing the biochemical changes in mineral metabolism accompanying the fall in kidney function, as presented in Lima, F, Mawad, H, El-Husseini, AA, Davenport, DL, Malluche, HH: Serum bone markers in ROD patients across the spectrum of decreases in GFR: Activin A increases before all other markers. Clinical nephrology, 91: 222-230, 2019 (Lima). This single-center study focused on establishing a link between activin A and bone biomarkers included a cross-sectional analysis of Ca, P, PTH, CTL, and FGF23 in 104 CKD stage 2-5D patients. Model validation was performed using an independent data set. Model fitting, simulation, and data analysis were performed using Matlab/Simulink 2019b (The Mathworks, Natick, MA). All data were digitized using WebPlotDigitizer (Ankit Rohatgi, San Francisco, Calif.).

In a second embodiment, the following parameters related to the newly added and modified model components were estimated using kidney function and bone mineral metabolism data from 5,496 participants averaging 6.1 observations per subject in the Chronic Renal Insufficiency Cohort (CRIC) study (Clinical Trials Identifier NCT00304148): parathyroid gland compartment (H⁻_{6.11}, H_{5.11}), FGF23 compartment (H⁺_{5.29}), 1αOH compartment (k_{9S}, H_{7.9}, and H⁻_{29.9}), renal phosphate reabsorption (Φ₅ᵤ and β₅ᵤ), and smooth muscle cell compartment (α_{5.30} and δ_{5.30}). In total, 23 model parameters were estimated. 33,451 data vectors were extracted from the CRIC cohort, the data vectors consisting of estimated GFR, calcium, phosphorus, CTL, PTH, and COOH-terminal FGF23. All units for calcium, phosphorus, PTH, and FGF23 are in systems international units. Model parameter estimation was performed using 10-fold cross-validation approach. Each fold contained 30,106 training vectors and 3,345 testing vectors. Parameter fitting was performed in ATLAB/Simulink 2020b (The MathWorks, Natick, Mass.) using constrained nonlinear least squares regression (Isqnonlin) with the trust-region reflective algorithm. Goodness of fit between the two models was tested by a t test of the resulting fitting of the models to the validation data.

Results

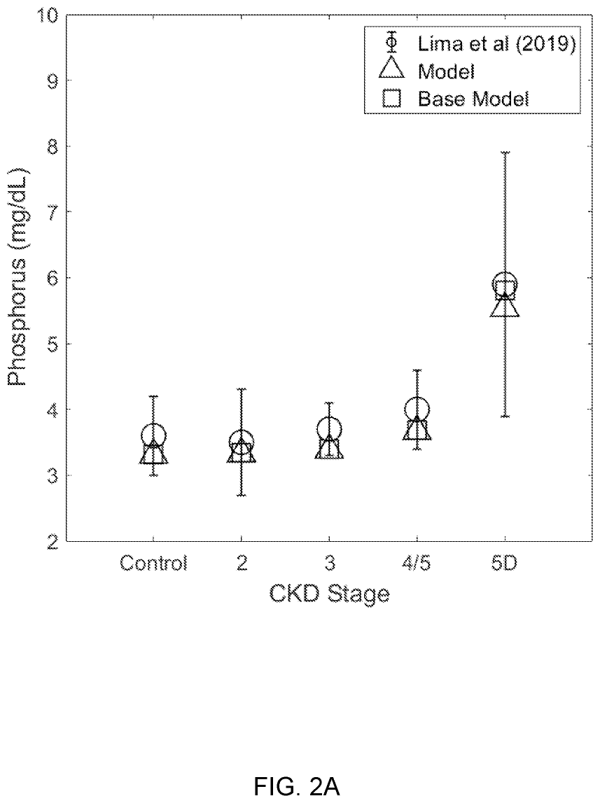

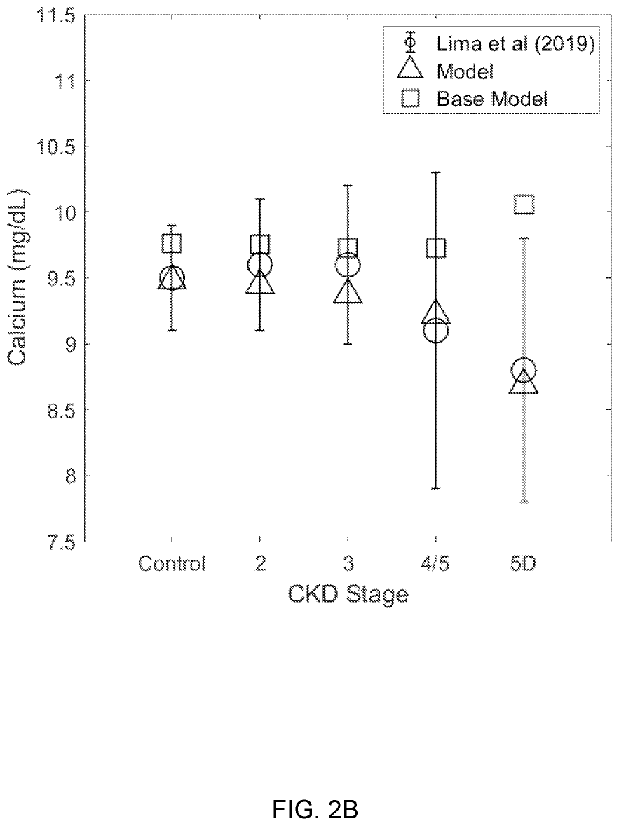

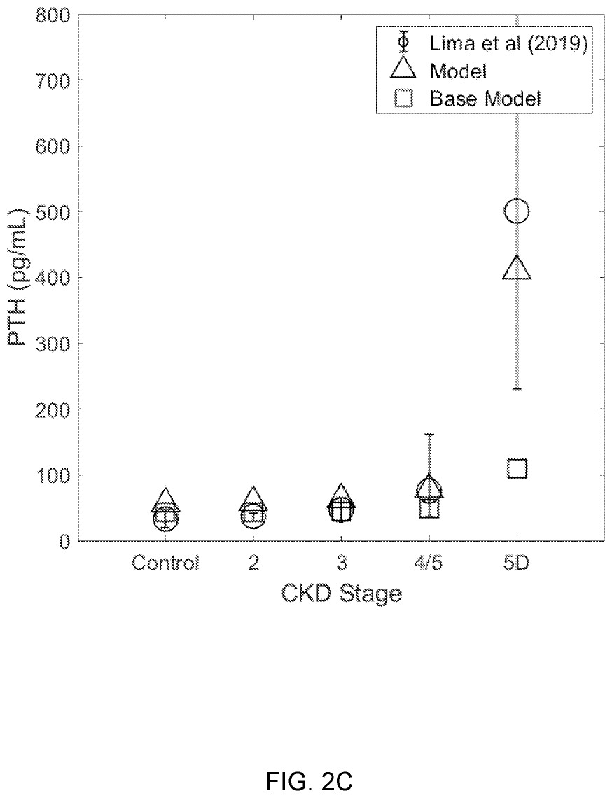

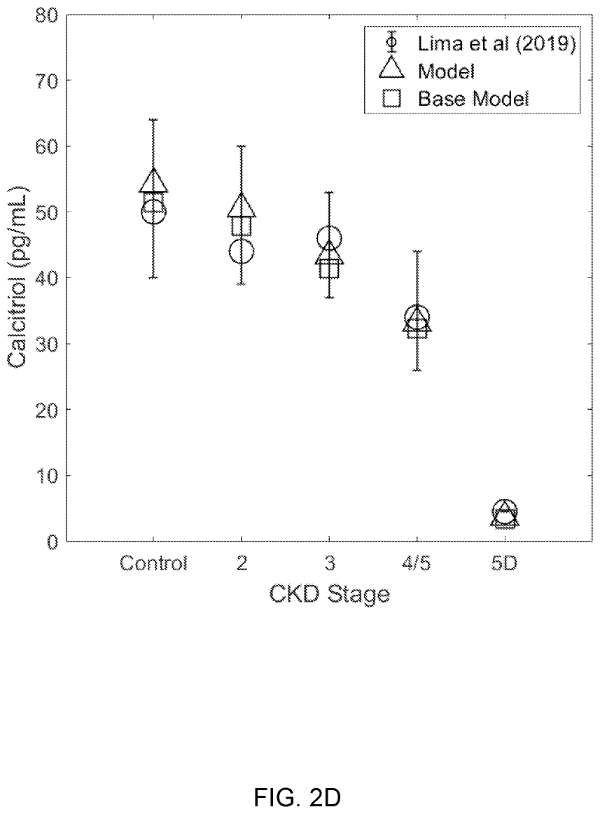

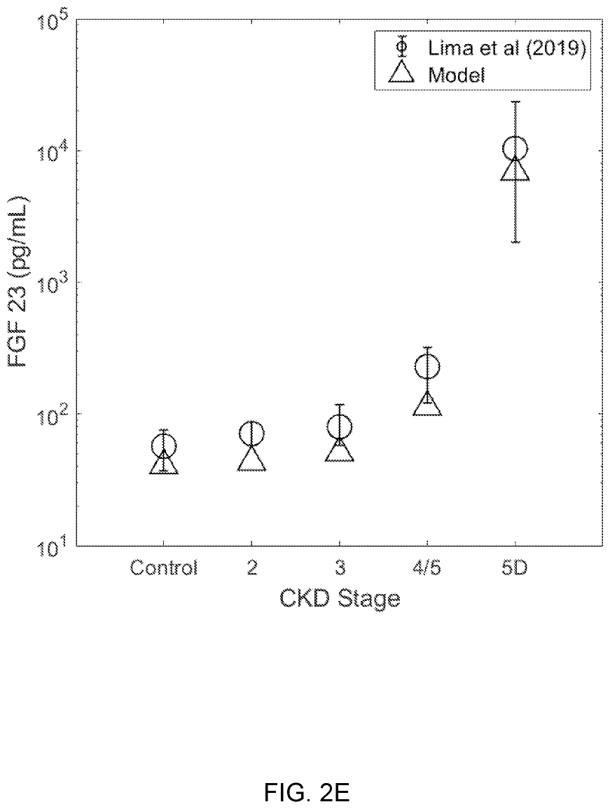

With respect to the first embodiment of the model, FIGS. 2A-2E show the changes in markers of mineral metabolism due to progression of CKD-MBD predicted by our model. These model predictions were compared to predictions generated by the base model and to data published by Lima. GFR values chosen to represent CKD stages Normal, 2, 3, 4/5, and 5D were 100, 75, 45, 15, and 5 mL/min/1.73m², respectively.

The model predictions are represented by point estimates whereas clinical data reported by Lima et al. are displayed as the mean±1 standard deviation or median±min/max where appropriate. Predictions of P concentration (FIG. 2A) at different level of kidney function performed by both models are consistent with the clinical data and show a small but steady increase in P through Stage 4 CKD followed by a more substantial increase as the kidney function declines to Stage 5D. Calcium concentrations (FIG. 2B) at different stages of CKD predicted by the model closely agree with the clinical data and show a progressive decrease in serum calcium as the kidney function declines through Stage 5D CKD. In contrast, calcium concentrations predicted by the original Peterson-Riggs model remain steady throughout all stages of CKD. PTH levels (FIG. 2C) predicted by both models remain constant through Stage 4 CKD. There is a considerable rise in PTH as patients enter Stage 5D CKD. This rise as predicted by the modified model more closely agrees with the clinical data, compared to the prediction made by the original Peterson-Riggs model. A steady decline in CTL concentration (FIG. 2D) with the declining kidney function observed in clinical data is accurate in both models. Finally, the log-transformed FGF23 concentrations (FIG. 2E) predicted at different levels of kidney function by the modified model closely match clinical data.

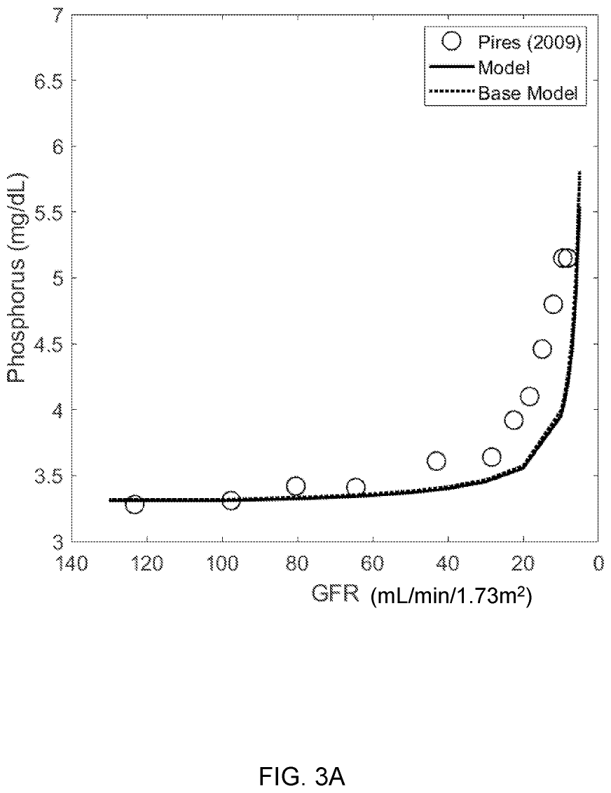

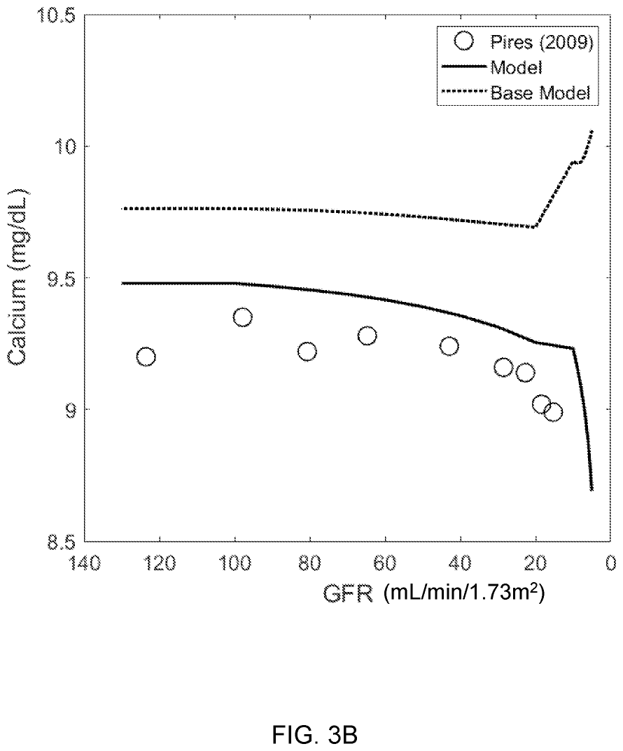

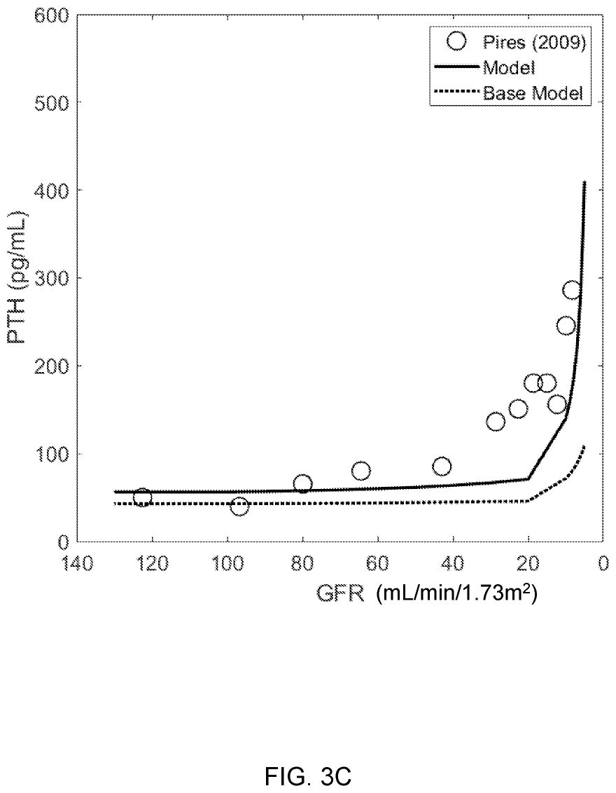

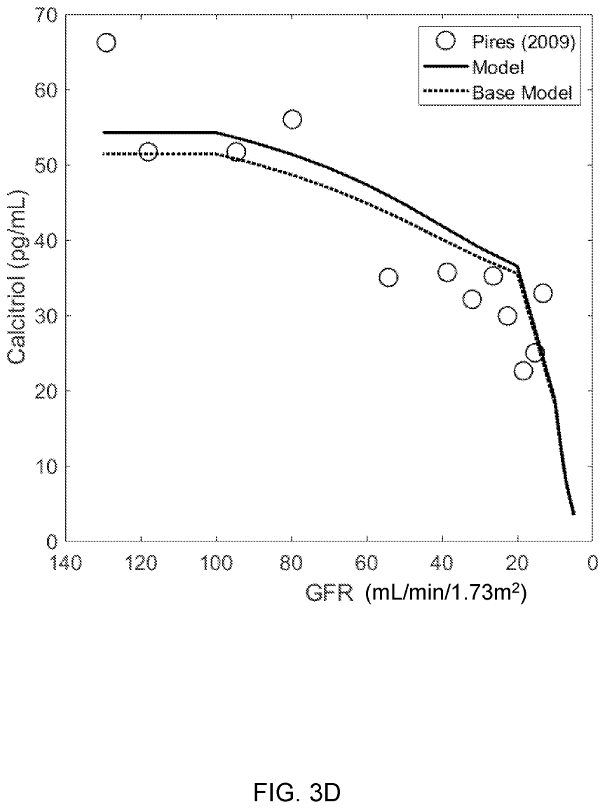

To validate the model, the values of the biochemical markers of mineral metabolism predicted by the first embodiment model and the base model were compared to an independent data set used by Raposo et al. Of note, this data set did not include dialysis patients. The first embodiment model and the base model were evaluated across the GFR range from 5 to 130 mL/min/1.73m². Model predictions were superimposed on point estimates extracted from validation data (FIGS. 3A-3D). Both models accurately predict the increase in P levels in late stage kidney disease (FIG. 3A). The models predict a rapid increase in P concentration below GFR of 20, compared to 25.1 reported in the data. The Ca levels predicted by the first embodiment model were slightly higher than the Ca levels reported by Raposo et al. but follow a similar trend of Ca concentration decreasing as the GFR decreases (FIG. 3B). On the other hand, the base model predicts a slight increase in Ca concentration as GFR drops below 20 mL/min/1.73m². PTH levels predicted by the first embodiment model follow the data (FIG. 3C). Both the first embodiment model and base model predict an increase in PTH as GFR decreases below 20 mL/min/1.73m², compared to GFR of 32.7 mL/min/1.73m² reported in published data. The increase in PTH predicted by the first embodiment model is more rapid than the one predicted by the base model and is more in line with the validation data. Predictions of CTL concentration by both models (FIG. 3D) closely match the validation data.

The movement of calcium from the bone to the blood and from the blood to the soft tissue predicted by the first embodiment model is displayed in FIG. 4. A two-fold increase in calcium flux into the soft tissue as the patient transitions to Stage 5D CKD likely represents soft tissue calcification and accounts for the observed decrease in total serum calcium.

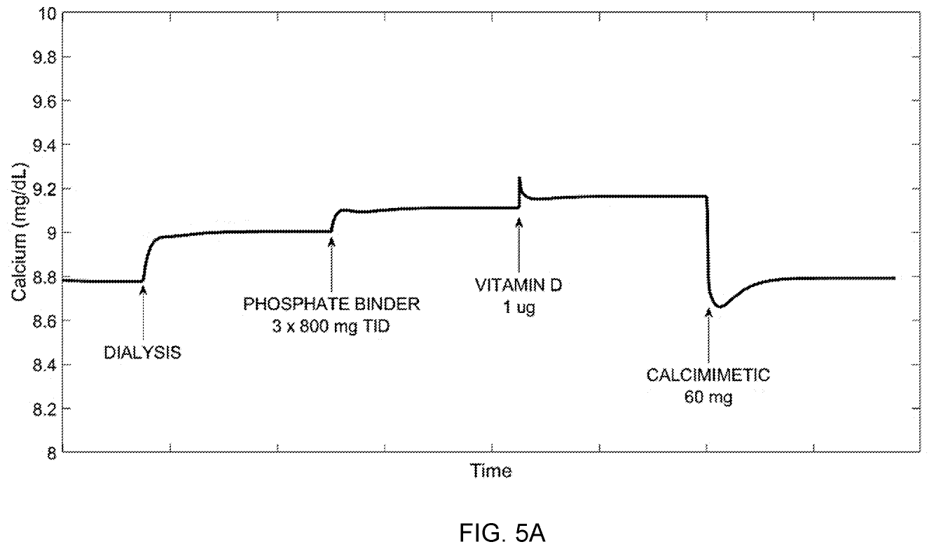

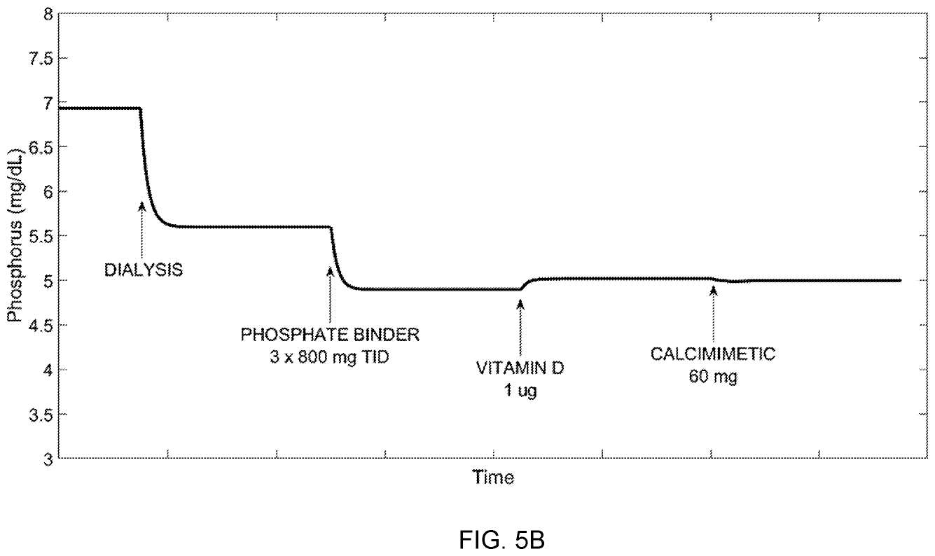

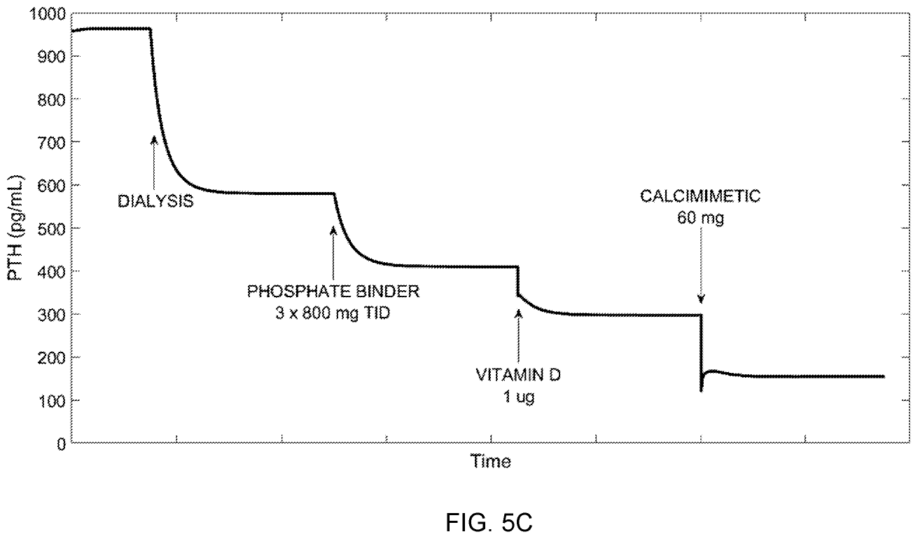

FIGS. 5A-5C show simulations of a hypothetical treatment regimen in a virtual patient with Stage 5D CKD. The initiation of three times a week hemodialysis with average P removal of 1000 mg per session results in a 2% increase in serum Ca, 20% decrease in P, and a 40% decrease in PTH. The addition of a phosphate binder (2400 mg TID) results in an additional 15% decrease in P and a 30% decrease in PTH without an observable change in Ca. The addition of a vitamin D analog (1 ug QD) decreases PTH by additional 25% without affecting Ca and P. Finally, the use of a calcimimetic (60 mg QD) results in a further 50% decrease in PTH. However, this effect is accompanied by a 4% drop in serum Ca.

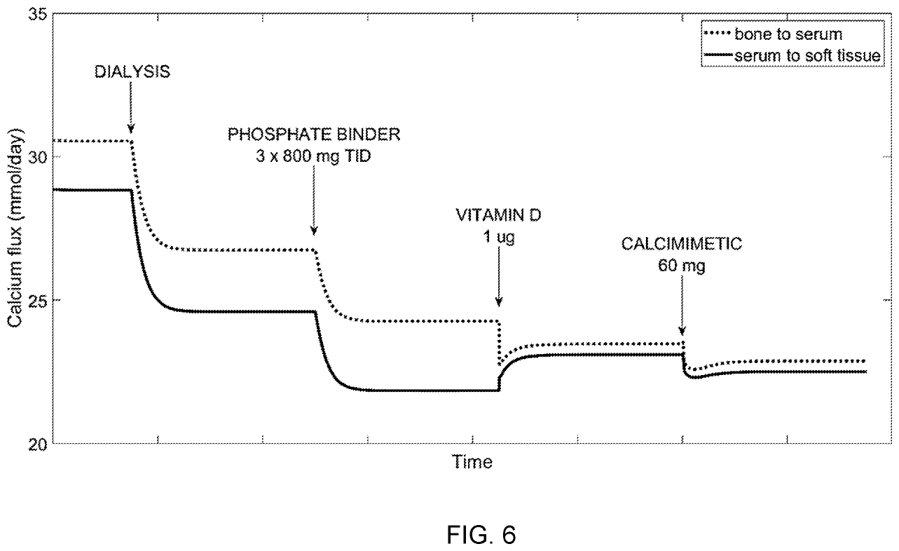

FIG. 6 shows simulated Ca fluxes from bone to blood and from blood to soft tissue in response to the hypothetical treatment regimen described above. The simulation of Ca flux between bone and serum shows that all four therapeutic interventions may have a beneficial effect on bone health by decreasing the Ca release from the bone. The simulation of Ca flux between serum and soft tissue shows that dialysis, the use of a P binder and a calcimimetic may decrease the Ca deposition in the soft tissue. From this simulation, it also appears that the addition of a Vitamin D analog may increase Ca flow into the soft tissue.

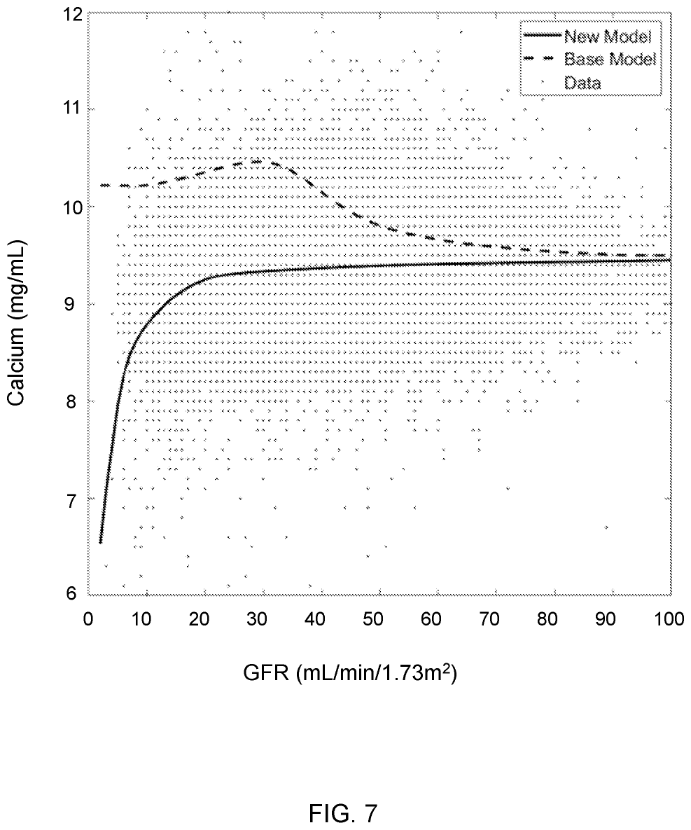

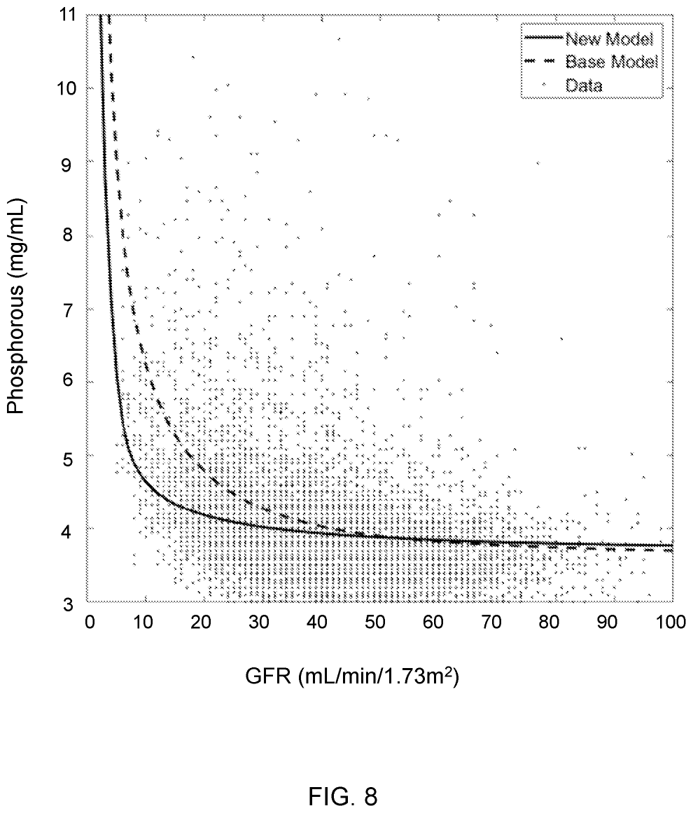

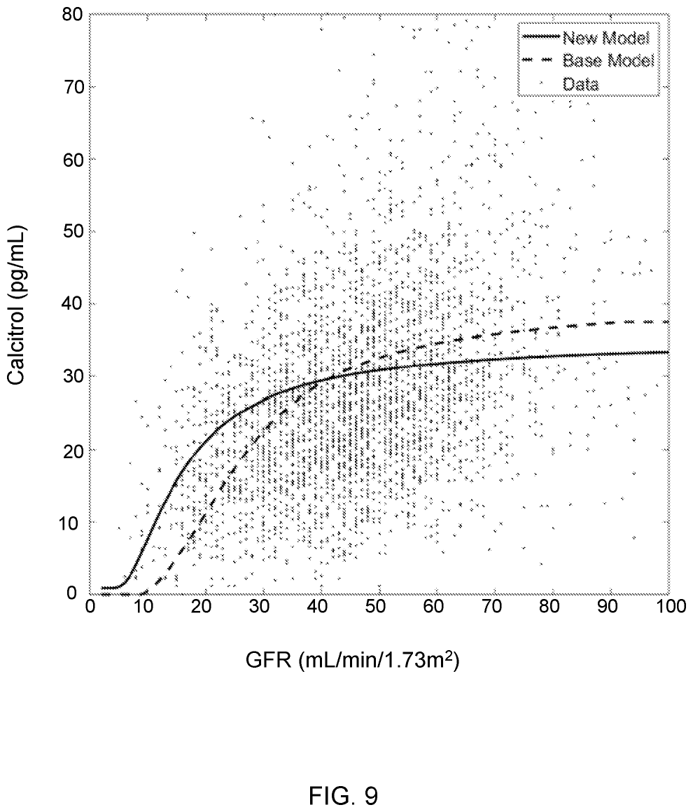

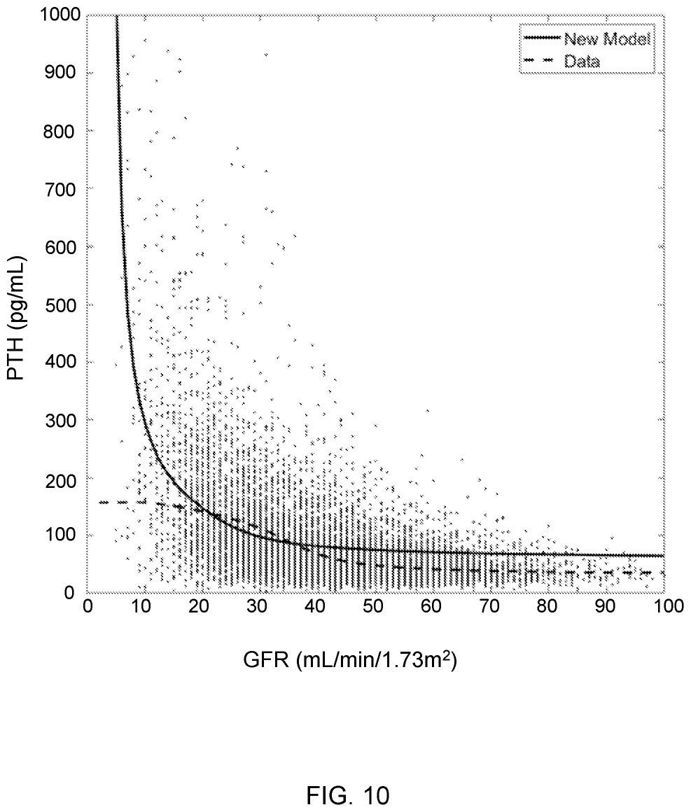

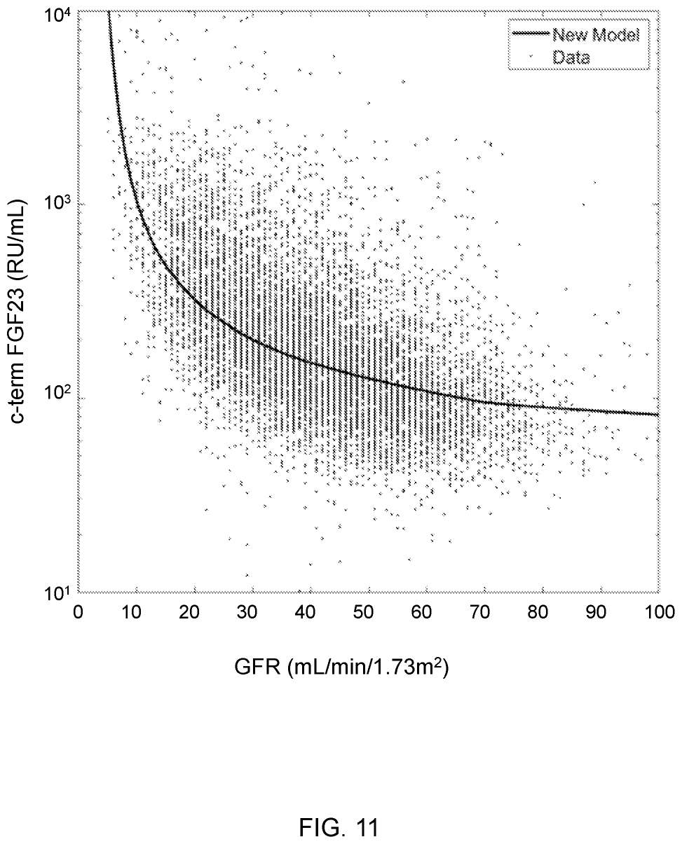

With respect to the second model, parameters were based on subjects in the CRIC with demographic data shown in Table 1. Results of model fitting are presented in Table 2 and FIGS. 7-11. Table 3 shows estimated model parameter means and their standard deviations across all 10 cross-validation folds for training and testing of the new model and the testing of the base model. The second embodiment model showed improvements over the base model based on the root mean square error for calcium, phosphorus, and PTH (Table 1). In FIGS. 7-10, the second embodiment model fit is displayed as a solid line and the base model as a dashed line. FIG. 7 shows the change in serum calcium concentration in the new model as a function of declining GFR predicted by the models superimposed on the CRIC data. Calcium concentration remained constant and showed a rapid decline when GFR decreased below 20 mL/min in the second embodiment model. Serum phosphorus concentration (FIG. 8) predicted by the model remained constant through GFR of 50 mL/min and showed a steady increase through GFR of 20 mL/min and a rapid increase once GFR dropped below 20 mL/min. Serum CTL concentration (FIG. 9) predicted by the second embodiment model remained constant through GFR of 30 mL/min and decreased rapidly to below 10 pg/mL as GFR dropped below 20 mL/min. The PTH level (FIG. 10) remained constant through GFR of 20 mL/min and increased exponentially thereafter. FIG. 11 shows a log-scale plot of change in COOH-terminal FGF23 with declining kidney function predicted by the model. The exponential increase in COOH-terminal FGF23 with decreasing GFR as shown in the data was accurately captured by the model. The base model failed to adequately describe the data for calcium and PTH.

TABLE 1
Demographic information for the CRIC data

| | eGFR Group at Enrollment | | | | | | | | |
|---|---|---|---|---|---|---|---|---|---|
| | <10 | 10-20 | 20-30 | 30-40 | 40-50 | 50-60 | 60-70 | 70-80 | >80 |
| N at enrollment* | 1 | 92 | 638 | 965 | 1277 | 1263 | 816 | 302 | 140 |
| Male/female | 0/100 | 55.4/44.6 | 51.4/48.6 | 51.6/48.4 | 57.3/42.7 | 59.9/40.1 | 57.8/42.2 | 59.6/42.2 | 56.4/43.6 |
| (% within category) | | | | | | | | | |
| Black/African | 100 | 45.7 | 43.9 | 43.5 | 44.4 | 45.3 | 43.3 | 46.4 | 44.3 |
| American (% within | | | | | | | | | |
| category) | | | | | | | | | |
| White (% within | 0 | 23.9 | 45.8 | 45.2 | 46.8 | 48.5 | 51.0 | 49.7 | 45.0 |
| category) | | | | | | | | | |
| Diabetic (% within | 0 | 69.6 | 56.4 | 56.1 | 54.8 | 49.2 | 42.4 | 47.0 | 43.6 |
| category) | | | | | | | | | |

| | eGFR Group in Modeling Vector | | | | | | | | |
|---|---|---|---|---|---|---|---|---|---|
| | <10 | 10-20 | 20-30 | 30-40 | 40-50 | 50-60 | 60-70 | 70-80 | >80 |
| n | 219 | 1876 | 4381 | 6352 | 7150 | 6684 | 4050 | 1792 | 909 |

*Two subjects were missing estimated glomerular filtration rate (eGFR) at enrollment.

TABLE 2
RMSE values for data used in parameter estimation
(training) and data held out for testing.

| | Second Embodiment | | Base Model | |
|---|---|---|---|---|
| | Model RMSE | | RMSE | |
| | Training | Testing | Testing | P Value |
| Calcium | 0.49 ± 0.001 | 0.49 ± 0.009 | 0.89 ± 0.017 | <0.001 |
| Calcitriol | 16.86 ± 0.214 | 16.68 ± 1.878 | 16.58 ± 1.749 | 0.25 |
| Phosphorous | 0.93 ± 0.008 | 0.925 ± 0.073 | 0.94 ± 0.062 | <0.001 |
| Parathyroid | 83.05 ± 1.293 | 82.85 ± 9.389 | 84.84 ± 11.167 | <0.001 |
| hormone | | | | |
| Log₁₀ | 0.35 ± 0.025 | 0.35 ± 0.030 | — | — |
| (FGF23) | | | | |

Values are means ± standard deviations. RMSE, root mean square error. P values are for comparison of the testing data in the new second embodiment model and the base model.

TABLE 3
Model parameter estimates and their standard deviations (SD)
derived from 10-fold cross-validation on the CRIC data

| Parameter | Estimate | SD |
|---|---|---|
| α_{5, 11} | 1.5178 | 0.0541 |
| δ_{5, 11} | 2.1041 | 0.0570 |
| γ_{5, 11} | 2.2864 | 0.1052 |
| α_{6, 11} | 9.0466 | 0.4387 |
| ρ_{6, 11} | 5.4692 | 0.2407 |
| δ_{6, 11} | 60.9574 | 2.9746 |
| γ_{6, 11} | 11.1803 | 0.6183 |
| ρ_{5, 29} | 0.0907 | 0.0163 |
| α_{5, 29} | 74.0469 | 4.6058 |
| δ_{5, 29} | 1.9380 | 0.0412 |
| γ_{5, 29} | 11.8004 | 0.5309 |
| α_{29, 9} | 0.7297 | 0.0480 |
| ρ_{29, 9} | 0.0016 | 0.0038 |
| δ_{29, 9} | 1.7157 | 0.1165 |
| γ_{29, 9} | 1.3928 | 0.0843 |
| α_{7, 9} | 2.1985 | 0.1121 |
| δ_{7, 9} | 1.4008 | 0.0798 |
| γ_{7, 9} | 0.1567 | 0.0731 |
| k₉ₛ | 7.1041 | 0.3489 |
| α_{5, 30} | 0.6392 | 0.0201 |
| δ_{5, 30} | 1.2038 | 00046 |
| Φ₅ᵤ | 1.2288 | 0.0240 |
| β₅ᵤ | 1.5178 | 0.0541 |

Discussion

Disclosed herein are two embodiments of a model for the Chronic Kidney Disease-Mineral Bone Disorder that faithfully simulates the pathophysiology over the full range of kidney function from normal to dialysis-dependent end stage kidney disease. Building upon the previously published base model of Riggs and Peterson additional key components of mineral metabolism are introduced that are characteristic of CKD and refined other aspects of their model. The new model shows improvements in calcium and PTH predictions that are useful when using the model to simulate drug dosing, and also includes the ability to predict FGF23 concentration and the movement of calcium and phosphorous into vascular tissue.

The ability of the model to reproduce the human condition was verified by comparing the predictions of the first embodiment of the model against the base model using published experimental data (Lima) (FIGS. 2A-2E), which were used to optimize the model parameters, and an independent data set, not used in the model building process (FIGS. 3A-3D), and by comparing the predictions of the second embodiment of the model with CRIC study data (FIGS. 7-11). The independent data set was presented in Pires, A, Adragao, T, Pais, MJ, Vinhas, J, Ferreira, HG: Inferring disease mechanisms from epidemiological data in chronic kidney disease: calcium and phosphorus metabolism. Nephron Clin Pract, 112: c137-147, 2009. The first embodiment model faithfully reproduces first data set, as expected. Few differences were observed between the model predictions and the validation data from Pires. This is not unexpected given the size of the datasets and the geographically different populations from which the data were obtained. The new model adequately reproduces the change in calcium, phosphorus, PTH, CTL, and FGF23 as kidney function decreases. Most importantly, the addition of the soft tissue compartment allowed for better predictions of serum calcium and can be used as a starting point to investigate the tissue calcification as a major morbidity associated with CKD-MBD. This model can now be used to hypothesize the impact of therapeutic manipulations on the attainment of the K/DOQI targets for Ca, P, and PTH as well as ways to modify the flux of calcium into the smooth tissue. Additionally, the model successfully recapitulates the expected responses to common therapeutic interventions such as the administration of active vitamin D or a calcium sensing receptor agonist.

The first addition to the base model was the incorporation of the effect of phosphorus on parathyroid gland hyperplasia. Parathyroid gland size in the base model is determined by the CTL concentration. To date, multiple studies have shown the relationship between phosphorus levels and parathyroid gland proliferation. This modification improved the model ability to predict the PTH increase in patients with end-stage kidney disease. With respect to the first embodiment, when compared to the base model, PTH concentrations increased in the stage 5 CKD dialysis group from a mean of 109 to 410 pg/mL, more accurately matching the observed data by Lima of 501 pg/mL.

The second addition to the base model was the incorporation of the soft tissue compartment as a site for mineralization. Impaired skeletal mineralization coupled with progressively severe vascular calcification is a key manifestation of CKD-MBD, with contributions from calcium, phosphorus, PTH, vitamin D, FGF23, and inhibitors of the Wnt signaling system. In line with the reported experimental data, development of soft tissue calcification was modeled due to increased serum phosphorus concentrations.

The addition of the soft tissue compartment did not have any observable effect on the predicted change in CTL level due to CKD-MBD progression. In contrast, the presence of the soft-tissue compartment yielded prediction of calcium and PTH changes with CKD-MBD progression that were consistent with published data. Also, while calcium concentration predicted by the base model increases with the progression of CKD-MBD accompanied by a minimal increase in PTH, the disclosed model predicts a decrease in calcium and a substantial increase in PTH as kidney function declines. The observed decrease in calcium can be explained by the greater calcium flux into the soft-tissue compartment precipitated by increasing phosphorus concentration. The difference in the observed increase in PTH between our model and the base model can be explained by the difference in serum calcium. Similar to the difference between serum calcium at CKD stage 5D, serum phosphorus predicted by the disclosed model was less than the base model, consistent with the movement of phosphorus into the soft-tissue compartment. The magnitude of the difference in predicted serum phosphorus concentrations, compared with the difference in calcium concentration, was consistent with the assumption of our model where the ratio of phosphorus to calcium movement into the soft-tissue compartment was 0.464.

The addition of FGF23 to the current model also represents a significant departure from the base model. Sustained elevation of FGF23 begins early in CKD in response to phosphorus retention and progressively increases as GFR declines, playing a critical role in the maintenance of phosphorus homeostasis but additionally suppressing CTL synthesis as predicted by the disclosed model.

Further additions to the base model were compartments that represented hemodialysis and smooth muscle and a pharmacodynamic effect of phosphate binders, vitamin D, and a calcimimetics. Simulation of dialysis treatment and the concurrent dosing of these therapeutic agents showed responses in calcium, phosphorous, and PTH that are consistent with clinical expectations. Here, the ability of the disclosed model to represent the behavior of physiologic markers, such as calcium and plosphorous fluxes between different compartments that are not measurable in a clinical care is a major strength of the model. Being aware of the effects of different treatment choices on the calcium and phosphorous transport between various compartments may be helpful in finding optimal treatment strategies for CKD-MBD. Treatment simulations with the model showed the expected beneficial effect of dialysis and phosphate binder use on the calcium, phosphorous, and PTH levels as well as the calcium flux from bone to serum (bone resorption) and the calcium flux from serum to soft tissue (vascular calcification). The addition of a vitamin D analog results in a slight increase of serum calcium and phosphorous and an observable decrease in PTH. In terms of calcium fluxes, bone resorption appears to be decreasing while vascular calcification becomes slightly intensified. The addition of a calcimimetic decreases PTH and calcium concentrations without affecting the P level. It also appears to slightly reduce bone resorption and vascular calcification.

This model can be used in a research setting. Specific questions about the effects of therapeutic agents on serum, bone, and soft tissue markers of CKD-MBD can be posed and the results of model simulations compared to human-derived data. A discrepancy between the predictions and the findings would suggest mechanisms not addressed by the model, potentially leading to the discovery of additional pathways for the development of CKD-MBD or unanticipated consequences of specific therapies. It is anticipated that individual requirements for changes in dialysis prescription, phosphate binders, vitamin D analogues, calcium sensing receptor agonists, even calcium chelators or antiresorptive agents will differ markedly. The model would allow investigators to identify patterns of responses, which could also generate alternative hypotheses for the development or progression of bone loss, soft tissue calcification, secondary hyperparathyroidism or other clinical consequences of CKD-MBD. Identifying differences in population response to different therapies would be possible. Prior studies have suggested that those individuals who are able to achieve the K/DOQI goals for CKD-MBD enjoy a superior survival. Application of this model to larger numbers of CKD and dialysis patients could increase the percentage of dialysis patients achieving the K/DOQI targets allowing investigators to study this question in a more prospective manner.

This model can also be used in the clinical setting to individualize and personalize the therapy of CKD-MBD at all stages of kidney disease including outpatient dialysis. The current approach to the treatment of CKD-MBD is empiric, driven by current incomplete understanding of the pathogenesis of CKD-MBD and our limited therapeutic armamentarium. The relative efficacies of the vitamin D analogues, the calcium sensing receptor agonists, and the phosphate binders for the control of CKD-MBD in the individual patient are not clinically apparent to the practitioner; thus, the choice of agent and the dose of agent are chosen according to each practitioner's habit. Likewise, adjustments in medication dose in response to changes in the parameters of mineral metabolism are based on prior experience and educated estimates of further response. The model presented here provides several advantages over this essentially trial and error approach. The model is capable of “learning” how each individual patient's parameters of mineral metabolism change in response to medication dosages, addition of medications, simultaneous use of several medications, and changes in dialysis prescription, enabling the model to predict ensuing responses to therapeutic changes and thus provide suggested dose adjustments. This model also allows for the introduction of additional CKD-MBD parameters and for the inclusion of additional therapies. Currently, only serum phosphate, calcium, and intact PTH are routinely evaluated to assess the CKD-MBD. An increasing number of mediators of bone and mineral metabolism including FGF23, sclerostin, DKK1, tartrate resistant acid phosphatase, osteocalcin, Receptor Activator of NFkappa B ligand (RANK ligand) and others are being identified and could potentially become clinically useful. These could also be included in the model relatively easily. Additional factors such as bone density and vascular calcification scores are potential future parameters to include.

Method for Creating a Dose Set for One or More Pharmacologic Agents

The above-described model is useful, among other purposes, in the simulation of the concentration of the chemicals, hormones, and cellular activity in patients with progressing kidney disease to the initiation of dialysis and then in the simulated treatment of with pharmacologic agents. In some embodiments, these pharmacologic agents are phosphate binders, vitamin D analogs, and calcimimetics. To create heterogeneity in response to pharmacologic agents we vary two model parameters: sensitivity of the calcium sensing receptor and phosphorus content of the diet. Other model parameters can be varied as needed to achieve more heterogeneity. To mimic the actual clinical experience, variable patient adherence to taking phosphate binders is also simulated. In contrast, vitamin D analogs and calcimimetics are usually administered via dialysis and patients typically adhere to dosing regimens for these drugs.

An agent derives drug doses from observed outputs, and can be represented by any function approximator architecture such as a deep neural network. In some embodiments, the agent is one or more dosing regimen program modules as described in U.S. Pat. No. 9,852,267. The model predicts observed and unobserved outputs from drug doses, and operates as described above. Unlike in U.S. Pat. No. 9,852,267, where model predictive control is used to determine dose recommendations, the instant invention preferredly uses a reinforcement learning agent.

A reinforcement learning agent broadly operates as a reward function which reinforces drug dose adjustments that move the concentrations of calcium, phosphorus, and PTH observed in the serum toward their target range. One can prioritize the attainment of one of the targets over the other by changing their importance in the reward function. In some embodiments, life-threatening low calcium concentrations are strongly penalized. Here, the agent receives observations regarding the computational model of the biological system, selects actions, namely, adjusting the dose set for one or more pharmacologic agents, in order to maximize reward by achieving an output physiological state of the computational model wherein the concentrations of calcium, phosphorus, and PTH observed in the serum are moved toward their respective target ranges. Several reinforcement learning agents, implemented as deep neural networks, have been implemented and validated as drug dosing agents.

The drug dosing agents receive six inputs: (1) difference between current phosphorus and the lower limit of the phosphorus target range, (2) difference between current serum phosphorus and the upper limit of the phosphorus target range, (3) difference between current serum calcium and the lower limit of the calcium target range, (4) difference between current serum calcium and the upper limit of the calcium target range, (5) difference between current serum PTH and the lower limit of the PTH target range, (6) difference between current serum PTH and the upper limit of the PTH target range. In other embodiments, additional input data may be provided. The drug dosing agents return three outputs as dosing recommendation ranging from −1 (decrease dose) through 0 (maintain current dose) up to 1 (increase dose) for the dose adjustment of the following pharmacologic agents: (1) phosphate binder, (2) vitamin D analog, (3) calcimimetic.

Two reinforcement learning algorithms were used to train these agents: Deep Q-Learning (DQN) and Deep Deterministic Policy Gradient (DDPG). Other reinforcement learning methods can also be used, such as, for example an actor-critic methods. Agents trained by DQN produce discrete dose recommendations (−1,0,1), whereas Agents trained by DDPG produce continuous dose recommendations (between −1 and 1). Continuous values for dose recommendations can be interpreted as strengths of the recommendation. For example a recommendation of −0.9 can be interpreted as a strong recommendation to decrease the dose of the drug, while a recommendation of −0.5 may indicate that maintaining the previous dose or decreasing it may be equally beneficial.

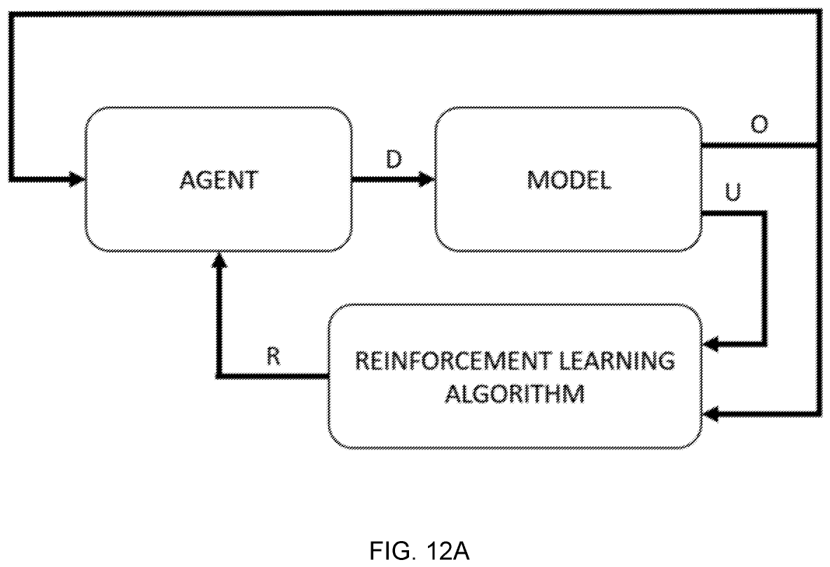

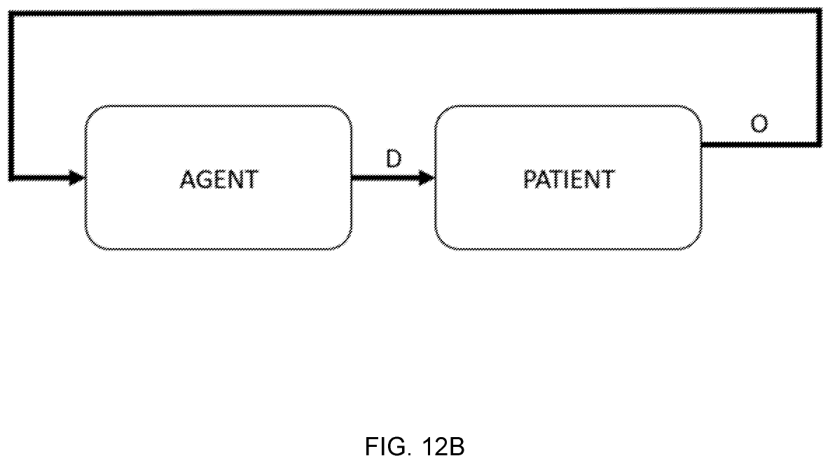

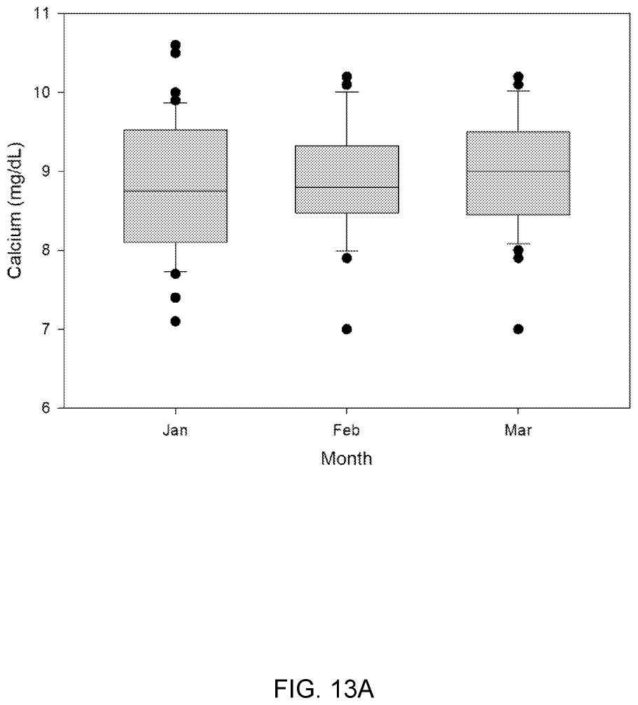

Referring now to FIGS. 12A and 12B, the figures represent reinforcement learning framework for off-line training of drug dosing agent (12A) and use of the agent with a patient (12B). In these figures, O=observed outputs, e.g. Ca, P, PTH concentrations; U=unobserved outputs (predicted by the model), e.g. calcium/phosphorus flux between serum, bone, soft tissue; osteoblast and osteoclast levels; R=reward function; D=drug doses, which in some embodiments are phosphate binder, vitamin D analogs, and calcimimetics.

In the agent training phase (i.e., off-line), as shown in FIG. 12A, the model simulates CKD-MBD disease progression and change in disease status in response to drug treatment as prescribed by the agent. Treatment adequacy is determined by the reward function. The reward function promotes treatment decisions that guide the disease status toward the desired outcome. The desired outcome is typically defined as Ca, P, and PTH maintained within user-defined target ranges. In some embodiments, user-defined target ranges are, for Ca, 8.4 mg/dL to 10.2 mg/dL, for P, 3.5 mg/dL to 5.5 mg/dL, and for PTH, 130 pg/mL to 600 pg/m L. In other embodiments, other target ranges may be used depending on the health and other characteristics of the patient or intended patient.

Ca, P, and PTH are periodically measured in CKD-MBD patients per current clinical standard of practice and are referred to as “observed outputs.” The desired outcome can also include quantities not typically measured but predicted by the model, such as Ca or P flux from bone to serum and Ca or P flux from serum to soft tissue, also referred to as “unobserved outputs”. These quantities represent two clinical outcomes of interest, namely bone resorption and vascular calcification whose risk should be minimized in CKD-MBD patients. Alternatively, other unobserved outputs predicted by the model can be included in the reward function depending on the other clinical outcomes of interest desired by the user. Agent training occurs by one of the reinforcement learning algorithms mentioned above. The goal of the agent is to select actions (doses or dose adjustments) which maximize the reward value over the simulation period, thereby maximize the likelihood of achieving the desired outcomes. The simulations are repeated until the reward value converges toward its maximum.

A trained Agent is deployed as a Clinical Decision Support System (on-line). Individual patient's Ca, P, and PTH levels are periodically queried (on-demand or automatically) from electronic health records and new dose (adjustments) recommendations for phosphate binder, vitamin D, and a calcimimetic are generated and sent to human operator (e.g., a physician) for approval. As a broad overview, the subject matter of the instant invention provides for improved dosing recommendations from an artificial intelligence agent, as the Agent interacts and trains with the model and learns optimal dosing patterns via reinforcement learning prior to generating new or adjusted dosing recommendations for a living patient.

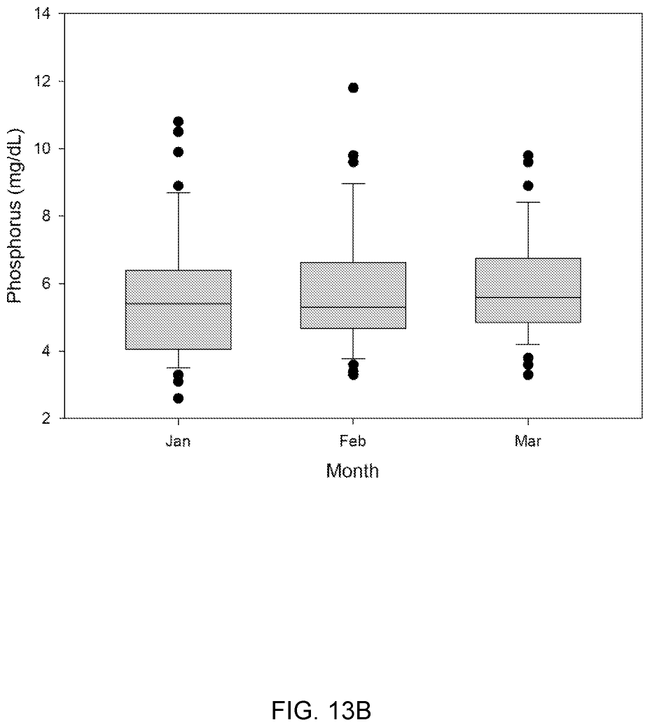

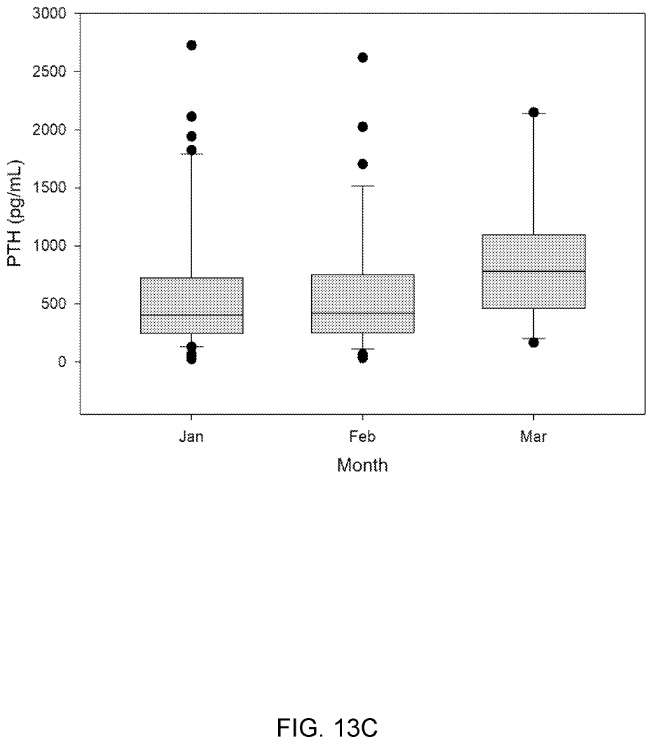

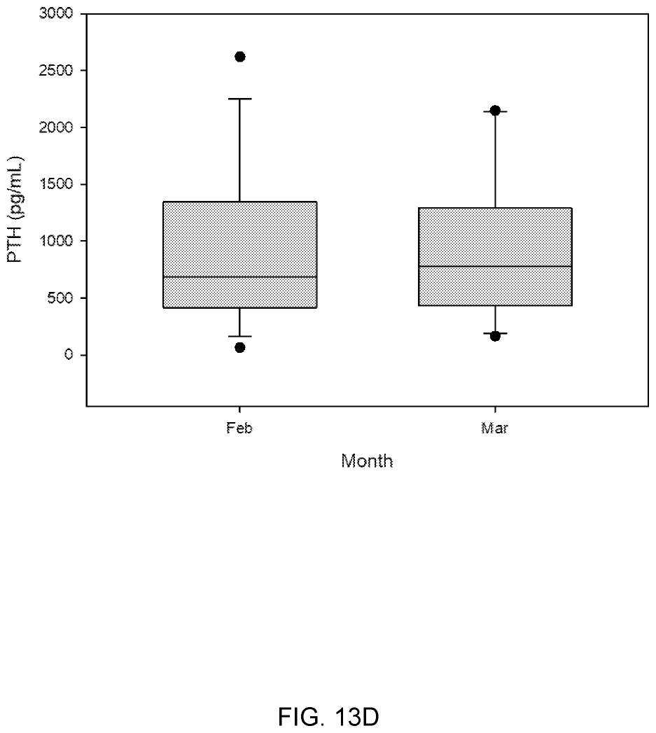

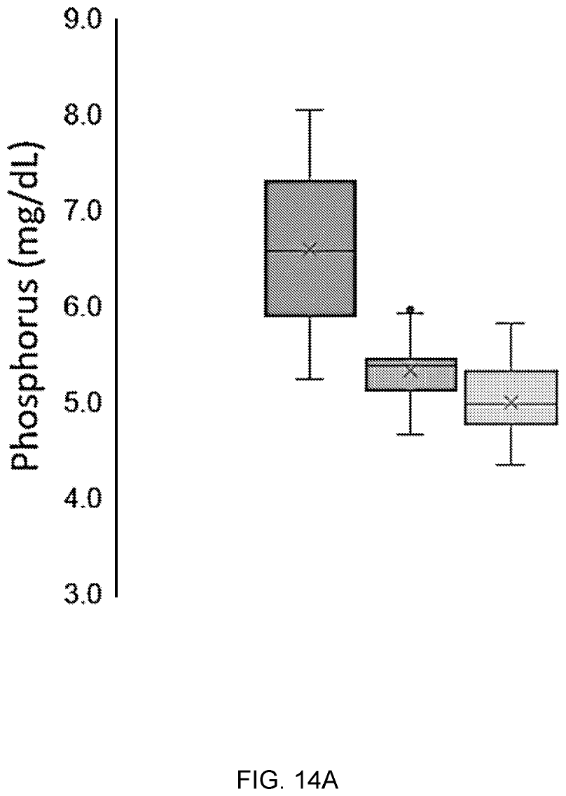

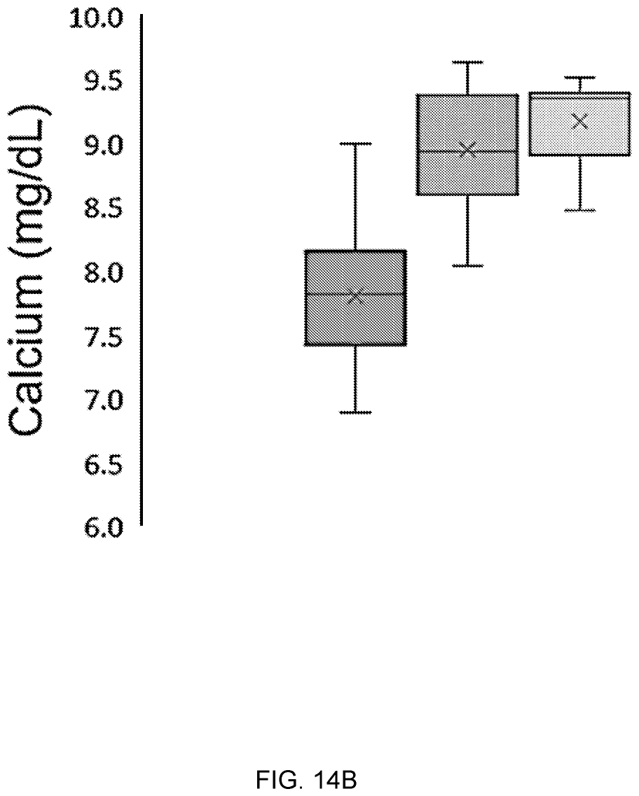

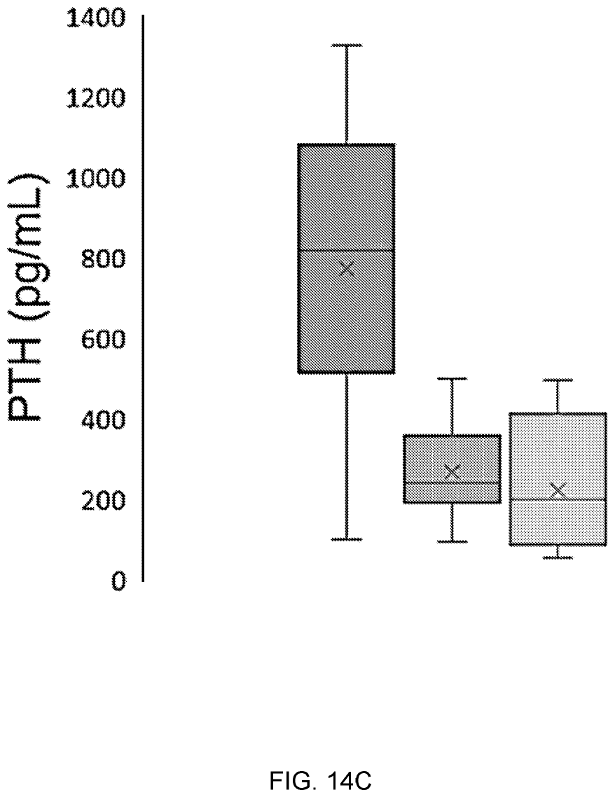

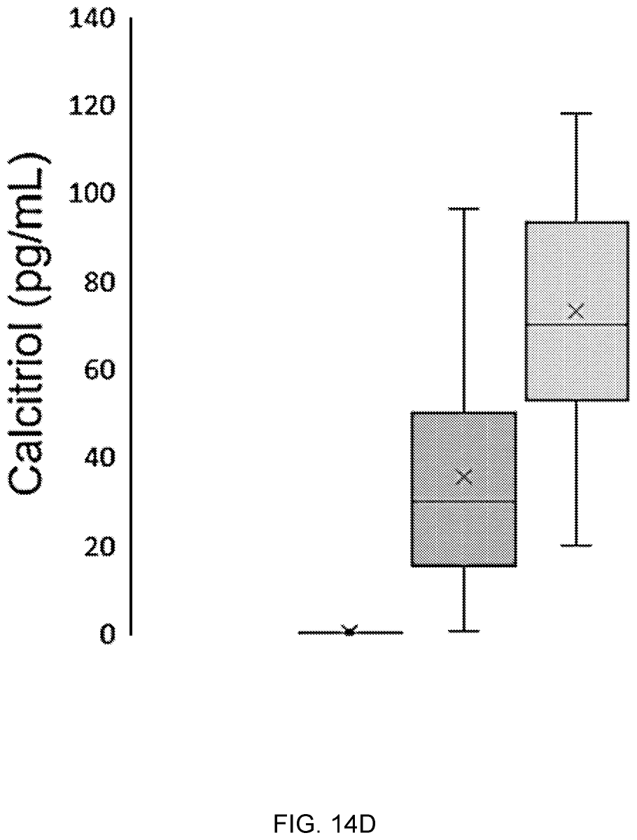

Agents' ability to maintain phosphorus between 3.5-5.5 mg/dL, calcium 8.5-9.9 mg/dL, and PTH 200-600 pg/mL were assessed. Based on the in silico assessment, a DDPG agent designated 407b was investigated in a quality improvement project in approximately 48 subjects. Beginning in January 2021 patients with calcium, phosphorus, and PTH measurements were presented to agent 407b and recommendations for phosphate binder, calcitriol, and cinacalcet (a calcimimetic) were made. This information was provided to the physician or nurse practitioner responsible for making dosing decisions and they could either accept, reject or modify the recommendation. Resulting calcium, phosphorus, PTH concentrations are shown in FIGS. 13A-13D. Data measured in January serve as the baseline prior to use agent 407b, while data in February and March reflect changes in patent serum values for calcium (FIG. 13A), phosphorous (FIG. 13B), and PTH (FIG. 13C) based on application of dose sets recommended by the drug dosing agent. Only a subset of twelve patients had PTH measured in both February and March, the results of which are shown in FIG. 13D. As is evident, PTH variability decreased between February and March.

Dose set recommendations generated by the disclosed invention are predicted to be improvements on dose set recommendations generated by a human physician. Using a generated data set, a deep neural network agent was trained from drug dosing data generated by an expert physician using supervised learning techniques. This agent was then trained using reinforcement learning (DDPG) as described above to achieve a target ranges of 3.5-5.5 mg/dL for phosphorous, 8.5-9.9 mg/dL for calcium, and 200-600 pg/mL for PTH. Referring now to FIGS. 14A-14D, the leftmost column shows the initial data, the middle column shows the concentration achieved by the agent trained via supervised learning, and the rightmost column shows the concentration achieved by the agent trained via reinforcement learning. In each case, the reinforcement learning agent outperformed the supervised learning agent, better achieving the target ranges in phosphorous (FIG. 14A), calcium (FIG. 14B), and PTH (FIG. 14C). Referring now to FIG. 14D, the recommended dose set for calcitriol, a vitamin D analog, generated by the supervised learning agent and reinforcement learning agent are compared in terms of concentration of the pharmacologic agent in the serum. As indicated by the leftmost column, the concentration of the agent was initially zero, increased by the supervised learning agent, and increased by a greater amount by the reinforcement learning agent.

The foregoing detailed description is given primarily for clearness of understanding and no unnecessary limitations are to be understood therefrom for modifications can be made by those skilled in the art upon reading this disclosure and may be made without departing from the spirit of the invention.

What is claimed is:

1) A system for personalized dosing of a pharmacologic agent comprising:
a data storage device;
a drug dosing agent stored on the data storage device, the drug dosing agent for determining a dose set for one or more pharmacologic agents;
a computational model of a biological system stored on the data storage device; a reinforcement learning algorithm stored on the data storage device;
and
a processing device in communication with the data storage device, the processing device configured to:
execute the drug dosing agent to determine the dose set for one or more pharmacologic agents;
execute the computational model to simulate the effects of the dose set, the computational model generating an output physiological state; and
execute the drug dosing agent to adjust the dose set for the one or more pharmacologic agents based at least in part on the output physiological state.

2) The system of claim 1, wherein the computational model is one of a quantitative systems pharmacology model and a systems biology model.

3) The system of claim 1, wherein the computational model is a model of chronic kidney disease.

4) The system of claim 1, wherein the computational model is a model of chronic kidney disease mineral bone disorder.

5) The system of claim 1, wherein the drug dosing agent is a deep neural network.

6) The system of claim 1, wherein the drug dosing agent adjusts the dose set for the one or more pharmacologic agents based in part on the output physiological state and based in part on physiological data from a subject.

7) The system of claim 1, wherein the computational model represents the biological system as a plurality of compartments, each compartment representing a tissue or organ, including a soft tissue compartment.

8) A method for providing personalized dosing of a pharmacologic agent to a patient, comprising:
obtaining a target range for an output physiological state;
determining, using a computer-implemented drug dosing agent, a dose set for a pharmacologic agent;
simulating, using a computational model of a biological system, effects of administering the dose set;
generating, using the computational model, the output physiological state based at least in part on the effect of the dose set;
repeating the determining, simulating, and generating steps until the output physiological state is within the target range, wherein the determining is based at least in part on the output physiological state; and
determining, using the computer-implemented drug dosing agent, a patient dose set for the pharmacologic agent.

9) The system of claim 8, wherein the computational model is one of a quantitative systems pharmacology model and a systems biology model.

10) The system of claim 8, wherein the computational model is a model of chronic kidney disease.

11) The system of claim 8, wherein the computational model is a model of chronic kidney disease mineral bone disorder.

12) The system of claim 8, wherein the drug dosing agent is a deep neural network.

13) The system of claim 8, wherein the output physiological state is a plurality of output physiological states, and wherein the target range is a plurality target ranges, each of the output physiological states having one target range.

14) The system of claim 8, wherein the pharmacologic agent is one of a phosphate binder, a calcimimetic, and vitamin D and analogs and metabolic precursors thereof.

15) The system of claim 8, wherein the output physiological state is calcium concentration and wherein the target range is 8.4 mg/dL to 10.2 mg/dL.

16) The system of claim 8, wherein the output physiological state is phosphorous concentration and wherein the target range is 3.5 mg/dL to 5.5 mg/dL.

17) The system of claim 8, wherein the output physiological state is parathyroid hormone concentration and wherein the target range is 130 pg/mL to 600 pg/m L.

18) The system of claim 8, wherein the pharmacologic agent modifies the output physiological state.

19) A data storage device having computer program instructions stored thereon that, when executed by a processor, cause the processor to perform the following instructions:
obtaining a target range for an output physiological state;
determining, using a computer-implemented drug dosing agent, a dose set for a pharmacologic agent;
simulating, using a computational model of a biological system, the effect of the dose set;
generating, using the computational model, the output physiological state based at least in part on the effect of the dose set;
repeating the determining, simulating, and generating steps until the output physiological state is within the target range, wherein the determining is based at least in part on the output physiological state; and
determining, using the computer-implemented drug dosing agent, a patient dose set for the pharmacologic agent.

20) A data storage device having computer program instructions stored thereon that, when executed by a processor, cause the processor to perform the following instructions:
simulate progression of chronic kidney disease metabolic bone disorder in a patient, the patient being represented by a plurality of compartments, each compartment representing a tissue or organ, wherein progression of chronic kidney disease metabolic disorder is simulated changes in concentrations of compounds in each compartment.

21) The data storage device of claim 20, wherein the compounds include at least one of fibroblast growth factor 23, calcium, phosphorous, and parathyroid hormone.

22) The data storage device of claim 20, wherein the plurality of compartments include a compartment representing smooth muscle cells.

23) The data storage device of claim 20, wherein the plurality of compartments include a compartment representing soft tissue.
